(19) United States
(12) Patent Application Publication
(10) Pub. No.: US 2003/0178022 A1
(43) Pub. Date: Sep. 25, 2003

(54) Pressurized metered dose inhaler (PMDI) actuators and medicinal aerosol solution formulation products comprising therse actuators

(75) Inventors: Rebecca Jayne Davies, Bath (GB); David Ganderton, Exeter (GB); David Andrew Lewis, Nr. Bath (GB); Brian John Meakin, Bath (GB)

Correspondence Address: David O. Seeley, P.O. Box 45862, SALT LAKE CITY, UT, 84145, US

(73) Assignee: Chiesi Farmaceutici S.p.A.

(21) Appl. No.: 10/326,565

(22) Filed: Dec. 19, 2002

Related U.S. Application Data
(60) Provisional application No. 60/348,888, filed on Jan. 15, 2002.

(30) Foreign Application Priority Data
Dec. 21, 2001 (EP) 01 130 521.6

(51) Int. Cl. A61M 11/00

(52) U.S. Cl. 128/200.23; 128/200.22

(57) ABSTRACT

The present invention relates to pressurized metered dose inhaler (pMDI) actuators having with laser-drilled orifices of novel dimensions, and to medicinal aerosol solution formulation products comprising these actuators. In particular, the present invention relates to the optimisation of the output characteristics of drug solution formulations in hydrofluoroalkanes (HFAs) by use of pMDIs with actuators with laser-drilled orifices of specific dimensions. Moreover, the actuators of the present invention allow the use of solution formulations with a high ethanol content and a high ratio of ethanol to active ingredients and thus, the use of poorly soluble active ingredients in solution formulations and allow the use of solution formulations which are substantially free of low volatility components.

### CROSS-REFERENCE TO RELATED APPLICATIONS

### 1. Cross Reference to Related Applications

The present application claims priority to United States provisional patent application Serial No. 60/348,888, filed Jan. 15, 2002, entitled “Pressurized Metered Dose Inhaler (pMDI) Actuators With Laser Drilled Orifices” which claims priority to European patent application Serial Number 01 130 521.6, filed Dec. 21, 2001, which application is incorporated herein by reference in its entirety.

### BACKGROUND OF THE INVENTION

### 2. The Field of the Invention

The present invention relates to pressurized metered dose inhaler (pMDI) actuators with laser drilled orifices and to medicinal aerosol solution formulation products comprising these actuators. In particular, the present invention relates to the optimisation of the output characteristics of drug solution formulations in hydrofluoroalkanes (HFAs) by use of pMDIs with actuators with laser drilled orifices of specific dimensions. Moreover, the actuators of the present invention allow the use of solution formulations with a high ethanol content and a high ratio of ethanol to active ingredients and thus, the use of poorly soluble active ingredients in solution formulations and allow the use of solution formulations with high ethanol content which are substantially free of low volatility components.

### SUMMARY OF THE INVENTION

The present invention relates to pressurized metered dose inhaler (pMDI) actuators having novel dimensions, and which are preferably formed using a laser.

The present invention also relates to medicinal aerosol solution formulation products involving the optimisation of the output characteristics of drug solution formulations in hydrofluoroalkanes (HFAs) by use of pMDIs with actuators with laser-drilled orifices of specific dimensions. Moreover, the actuators of the present invention allow the use of solution formulations with a high ethanol content and a high ratio of ethanol to active ingredients and thus, the use of poorly soluble active ingredients in solution formulations and allow the use of solution formulations which are substantially free of low volatility components.

### BRIEF DESCRIPTION OF THE DRAWINGS

To further clarify the advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a cutaway side view of a pressurized metered dose inhaler.

FIG. 2 depicts an actuator nozzle block.

FIG. 3 is a sectional view taken along lines 2-2 of FIG. 2.

FIG. 4 is an enlarged reversed view of the portion of FIG. 3 indicated by the circled reference to FIG. 4.

FIG. 5 is a front view of a nozzle block in accordance with the present invention.

FIG. 6 is a sectional view taken along the line A-A of FIG. 5.

FIG. 7 is a graph depicting the relationship of spray duration to orifice diameter.

### DETAILED DESCRIPTION OF THE PREFERRED EMBODIMENTS

The pharmaceutical solution formulations in hydrofluoroalkanes used in the present invention may be filled into canisters suitable for delivering pharmaceutical aerosol formulations. Canisters generally comprise a container capable of withstanding the vapour pressure of the HFA propellant, such as plastic or plastic-coated glass bottle or preferably a metal can, for example a stainless steel can or aluminium can which is preferably anodised, organic coated, such as lacquer-coated and/or plastic coated (WO00/30608 of the applicant), which container is closed with a metering valve. The metering valves comprising a metering chamber are designed to deliver a metered amount of the formulation per actuation and incorporate a gasket to prevent leakage of propellant through the valve. The gasket may comprise any suitable elastomeric material such as for example low density polyethylene, chlorobutyl, black and white butadiene-acrylonitrile rubbers, butyl rubber and neoprene. Thermoplastic elastomer valves as described in WO92/11190 and valves containing EPDM rubber are especially suitable. Suitable valves are commercially available from manufacturers well known in the aerosol industry, for example, from Valois, France (e.g. DF10, DF30, DF31, DF60), Bespak plc UK (e.g. BK300, BK356, BK357) and 3M-Neotechnic Ltd. UK (e.g. Spraymiser™).

Valve seals, especially the gasket seal, and also the seals around the metering chamber, will preferably be manufactured from a material which is inert to and resists extraction into the contents of the formulation, especially when the contents include ethanol.

Valve materials, especially the material of manufacture of the metering chamber, will preferably be manufactured of a material which is inert to and resists distortion by contents of the formulation, especially when the contents include ethanol. Particularly suitable materials for use in manufacture of the metering chamber include polyesters eg polybutyleneterephthalate (PBT) and acetals, especially PBT.

A valve stem extends from the metering valve and acts as a conduit to pass the metered dose into a nozzle block situated in the actuator body, in which the valve stem is seated.

Materials of manufacture of the metering chamber and/or the valve stem may desirably be fluorinated, partially fluorinated or impregnated with fluorine containing substances in order to resist drug deposition.

Each filled canister is conveniently fitted into a suitable channelling device prior to use to form a metered dose inhaler for administration of the medicament into the lungs or nasal cavity of a patient. Suitable channelling devices comprise, for example a valve actuator and cylindrical or cone-like passage through which medicament may be delivered from the filled canister via the metering valve to the nose or mouth of a patient e.g. a mouthpiece actuator.

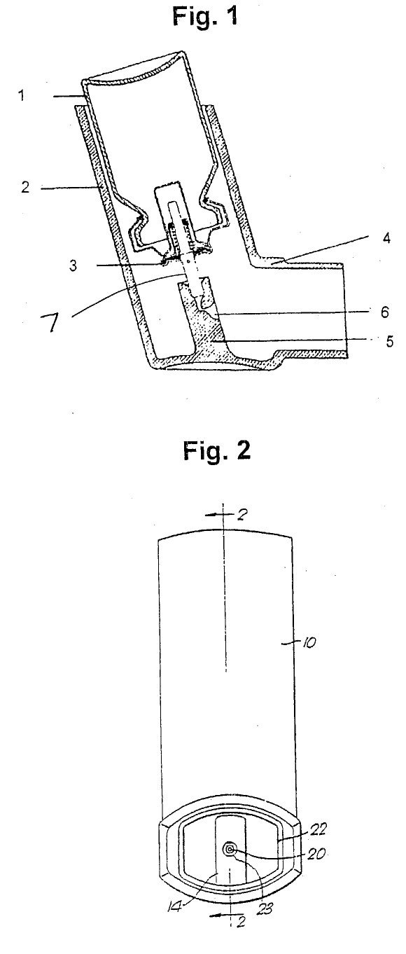

In a typical arrangement (FIG. 1) the valve stem 7 is seated in a nozzle block which comprises an actuator insert 5, which comprises an actuator orifice 6 leading to an expansion chamber. Conventional pressurized metered dose inhaler actuators have variable actuator orifice diameters from 0.25 to 0.42 mm and a length from 0.30 to 1.7 mm. In other types of actuators the lengths can vary. International Patent Application WO01/19342 discloses actuator orifice diameters in the range of 0.15 to 0.45 mm, particularly 0.2 to 0.45 mm. According to this prior art reference it is advantageous to use a small diameter e.g. 0.25 mm or less, particularly 0.22 mm since this tends to result in a higher FPM (fine particle mass) and lower throat deposition. Moreover it is stated that 0.15 mm is also particularly suitable. However, this prior art reference does not disclose how to obtain actuator orifices of less than 0.2 mm. The examples only relate to pMDIs having actuator orifices of 0.22 mm, 0.33 mm and 0.50 mm. Thus, although referring in general to small actuator orifice diameters of less than 0.2 mm, the prior art does not provide a solution how to obtain such small orifices with a high precision, i.e. with tightly controlled tolerances.

WO 01/58508 discloses an actuator for a metered dose inhaler containing a liquefied propellant and a medicament. The actuator comprises a nozzle block having a fluid flow path extending therethrough, the fluid flow path defined by an internal chamber having an inlet and an outlet; the outlet being defined in a portion of said nozzle block and comprising an exit channel extending therethrough. The exit channel has a narrow portion wherein the diameter of the channel is 0.3 mm or less, the narrow portion being 0.5 mm or less in length; and the narrow portion optionally including a constriction having a diameter of less than 0.3 mm. According to WO 01/58508, the increased degree of material deposition typically encountered with the use of nozzle orifices having a diameter of 0.3 mm or less may be reduced to a level at or below that experienced with larger diameter nozzles while still producing the high fine particle fractions achievable through using small diameter orifice nozzles (0.3 mm or less). This is accomplished by limiting the length of the portion of the nozzle channel which is 0.3 mm or less in diameter to 0.5 mm or less in length.

WO 99/55600 discloses a medicinal aerosol product having a blockage resistant metered-dose valve with a metal valve stem, particularly for use with CFC-free solution formulations using hydrogen containing propellants, such as 134a and/or 227, and ethanol. Moreover, a metered dose inhaler comprising an actuator and an aerosol product is disclosed. The actuator comprises a nozzle block and a mouth piece, the nozzle block defining an aperture for accommodating the end of the valve stem and an orifice in communication with the aperture directed towards the mouth piece, the orifice having a diameter of less than 0.4 mm, preferably about 0.3 mm.

Metered dose inhalers are designed to deliver a fixed unit dosage of medicament per actuation shot or “puff”, for example in the range of 25 to 250 μg medicament per puff, depending on the metering chamber volume used.

These and other objects and features of the present invention will become more fully apparent from the following description and appended claims, or may be learned by the practice of the invention as set forth hereinafter.

In the accompanying drawings, FIG. 1 shows a conventional pressurized metered dose inhaler comprising a canister 1, an actuator 2, a metering valve 3 with a valve stem 7, an oral tube 4, and a nozzle block comprising an actuator insert 5 and an actuator orifice 6.

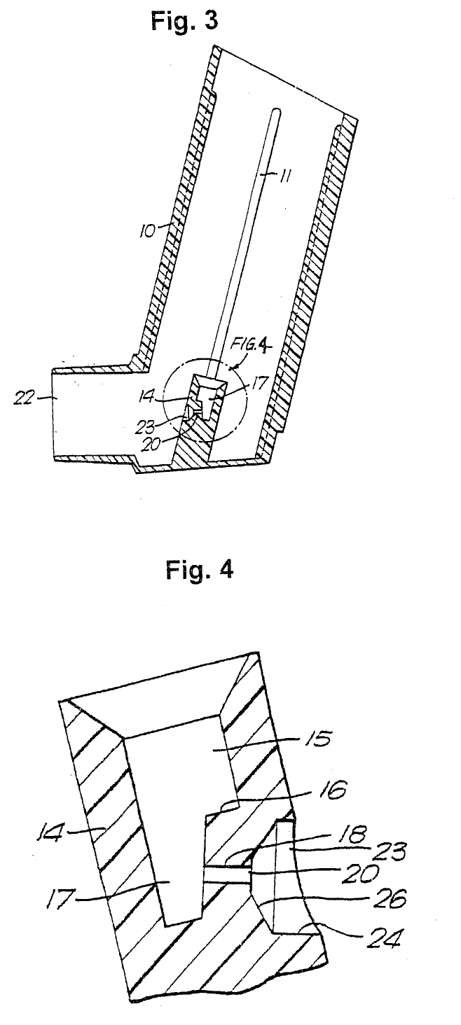

FIGS. 2, 3 and 4 show a conventional actuator nozzle block. FIG. 3 is a section on line 2-2 of FIG. 2, and FIG. 4 is an enlarged reversed view of the circled part of FIG. 3.

Referring to the figures, a conventional pressurized metered dose inhaler consists of a body portion 10 of an actuator into which a pressurized canister 1 containing a medicinal aerosol solution formulation may be inserted, and located by means of ribs 11.

A nozzle block 14 of the body portion 10 has a bore 15 which receives the valve stem 7 of the canister 1. The end of the stem bears on a step 16 within the base so that compressing the body portion 10 and canister 1 together opens the valve 3 and causes the discharge under pressure of a single measured quantity of the drug in its carrier medium.

The dose passes down a passage 17 in the nozzle block 14, through a conduit 18 (with an actuator orifice length), e.g. a parallel-bore conduit to a discharge nozzle 20 (with an actuator orifice diameter), and thence through a mouthpiece 22 of the body portion 10 of the actuator.

The shape and direction of the discharge plume and the dispersion of the droplets or particles therein are critical to effective administration of a controlled dose to the patient.

Conventionally the discharge nozzle 20 is positioned in a cylindrical recess 23 in the nozzle block 14 having a parallel sided portion 24 and a frusto-conical base 26. In order for the patient to insert the mouthpiece at the correct orientation for discharge of the spray whilst at the same time holding the body portion 10 of the actuator and the canister 1 at a convenient angle, the axis of the mouthpiece 22 is inclined at an obtuse angle of about 105 degrees to that of the body portion 10 of the actuator and nozzle block 14. Because of this geometry, the conical recess is not perpendicular to the surface of the nozzle block 14, resulting in the parallel sided portion 24 being shorter on one side than the other.

The dimension of the discharge nozzle 20 (actuator orifice diameter) and the recess 23 are such that the discharge plume dose not impinge directly upon the sides of the recess 23.

A problem with known inhaler spray nozzles is that of adequately matching the dimensions of the conduit 18 (actuator orifice length) and nozzle 20 (actuator orifice diameter) to the particular drug formulation and carrier-propellant. Different drugs have different flow and dispersion characteristics (particularly as between suspensions wherein drug particles are dispersed in the formulation and solutions wherein the drug is completely dissolved in the formulation) and it is often difficult to achieve the optimum balance between the plume shape, total dose volume and plume duration.

It has been disclosed (Lewis D. A. et al., Respiratory Drug Delivery VI, 363-364, 1998) that using commercially available actuators for delivering solution formulations of aerosol pressurized with HFA, the reduction in the orifice diameter from 0.42 to 0.25 mm induces an increase in the fine particle dose (FPD) of the aerosol produced.

FPD, which provides a direct measurement of the aerosol particles considered suitable for deposition and retention in the respiratory tract, is calculated as the mass of the particles deposited from stage 3 to filter (particles with an aerodynamic diameter less than 4.7 μm) of the Andersen Cascade Impactor.

The aerodynamic particle size distribution of an aerosol formulation is characterised using a Multistage Cascade Impactor according to the procedure described in European Pharmacopoeia 2nd edition, 1995, part V.5.9.1, pages 15-17. Generally an Andersen Cascade Impactor (ACI) is utilised. Deposition of the drug on each ACI plate is determined by high performance liquid chromatography (HPLC). Mean metered dose is calculated from the cumulative deposition in the actuator and ACI stages; mean delivered dose is calculated from the cumulative deposition in the ACI. Mean respirable dose (fine particle dose, i.e. FPD) which provides a direct measurement of the aerosol particles considered suitable for deposition and retention in the respiratory tract, is obtained from the deposition on Stage 3 (S3) to filter (AF) corresponding to particles ≦4.7 μm. Smaller particles, with an aerodynamic diameter ≦1.1 μm correspond to the fraction obtained from the deposition on Stage 6 to filter.

The FPD can also be expressed as a percentage of the ex-valve dose or recovered dose (i.e. Fine Particle Fraction: FPF_{≦4.7 μm} or FPF_{≦1.1 82 m}). Shot weights are measured by weighing each canister before and after the actuation.

HFA solution formulations usually contain a co-solvent, generally an alcohol and specifically ethanol, to dissolve the active ingredient in the propellant. Depending on the concentration and solubility characteristics of the active ingredient, the concentration of solubilization agent (e.g. ethanol) can increase. Large amounts of ethanol increase, proportionally to their concentration, the velocity of the aerosol droplets leaving the actuator orifice. The high velocity droplets extensively deposit into the oropharyngeal tract to the detriment of the dose which penetrates in the lower airways (i.e. respirable fraction or fine particle fraction (FPF)).

The technical problem underlying the present invention is to provide pressurized metered dose inhaler actuators with an optimisation of the output characteristics of drug solution formulations in hydrofluoroalkanes (HFAs). In particular, it is a technical problem underlying the present invention to provide actuators with an extremely efficient atomisation also with HFA solution formulations containing high levels of ethanol and high ratios of ethanol to active ingredient, i.e. showing a fine particle fraction (i.e. particles with a diameter smaller than 4.7 μm) of at least 50%, preferably at least 60% and more preferably at least 70% and an optimum balance of the plume shape, total dose volume and the plume duration. Moreover, blockage and clogging problems due to material deposition should be avoided.

These technical problems have been solved by actuators and a medicinal aerosol solutions as described and claimed herein.

According to the present invention an actuator is provided with an actuator orifice having a diameter below 0.20 mm over the entire actuator orifice length, preferably in the range from 0.10 to 0.20 mm, more preferably 0.11 to 0.18 mm and in particular from 0.12 to 0.18 mm over the entire actuator orifice length, wherein a diameter of 0.12 mm, 0.14 mm, 0.16 mm and 0.18 mm is particularly preferred. The orifice diameter can be different at the inlet and at the outlet of the actuator orifice, however, has to be in the given ranges over the entire actuator orifice length. Preferred orifice diameter combinations inlet/outlet (mm) are 12/18, 18/12, 14/18, 18/14, 16/18, 18/16, 12/16, 16/12, 14/16 and 16/14. The small actuator orifice diameters are obtained by using a laser to drill the actuator orifices. The advantages of using a laser to drill the actuator orifices include, very high precision down to a few microns, smooth interior bore, tightly controlled taper and dimensional tolerances, entry angle holes down to 10 degrees and minimal heat damage. Thus, the present invention provides an alternative to existing moulding techniques and provides pMDI actuators with very small actuator orifice diameters with tightly controlled tolerances which is necessary to be able to provide tightly controlled reproducibility of the unit dosage of medicament per actuation.

In addition to the actuator orifice diameter, the actuator orifice length is an essential feature according to the present invention. Preferably, the actuator orifice has a length in the range from 0.60 mm to 1.00 mm, in particular from 0.60 mm to 0.80 mm.

For example a copper vapour laser (CVL) (Oxford Lasers ltd.) can be used to produce actuators with tightly controlled tolerances on orifice diameter and length.

The dimensions of the actuator orifices are checked using a Mitntoyo TM WF20X microscope and Dolan-Jenner Fiberlite.

According to the present invention, specific combinations of actuator orifice diameter and length provide actuators with improved actuator blockage/device clogging characteristics, in particular in combination with drug solution formulations in hydrofluoroalkanes having a high ethanol and/or water content and a high ratio of ethanol to active ingredient and having a low content or being devoid of low volatility components such as glycerol. Water can be present as a co-solvent in an amount of up to 5% by weight. Furthermore, the presence of water can improve the chemical stability of certain active ingredients.

Actuator orifice length of the nozzle blocks of the present invention refers to the distance between the external face (outlet) and the internal surface (inlet) which due to the design of the nozzle blocks are parallel.

According to the present invention, a medicinal aerosol solution formulation containing an active ingredient, preferably a corticosteroid selected from beclometasone dipropionate, budesonide, dexbudesonide, ciclesonide, fluticasone propionate and mometasone propionate, and a β₂-agonist selected from formoterol, salmeterol xinafoate and TA 2005, a hydrofluorocarbon propellant such as HFA 134a, HFA 227 and mixtures thereof, ethanol as a cosolvent in an amount of at least 7% by weight, preferably at least 15% by weight and up to 20 or 25% by weight or more, based on the solution formulation, and in a ratio of ethanol:active ingredient of at least 20:1, preferably 30:1 and more preferably of at least 35:1 by weight, and optionally a low volatility component, such as glycerol, propyleneglycol, polyethyleneglycol and isopropylmyristate in an amount of from 0 to 0.5% by weight, based on the solution formulation is used in a pressurised metered dose inhaler, comprising a canister equipped with a metering valve and the actuator of the present invention as defined above.

Other preferred solution formulations contain a medicament which could take advantage from a pulmonary delivery to produce a systemic therapeutic effect.

The use of the above-described medicinal aerosol solution formulations of the present invention in a pressurized meter dose inhaler comprising an actuator of the present invention as described above results in a medicinal aerosol solution formulation product providing an aerosolised medicament showing a fine particle fraction of at least 50% and an optimum balance of the plume shape, total dose volume and the plume duration. Moreover, blockage and clogging problems due to materials depositions are avoided by a solution formulation being substantially free of low volatility components, i.e. containing 0 to about 0.5%, preferably 0 to about 0.3% and in particular 0 to 0.1% by weight of a low volatility component such as glycerol. The use of these kinds of solution formulations results in particles with a MMAD (Mass Median Aerodynamic Diameter) ≦2. Thus, the present invention provides a medicinal aerosol solution for a medicinal aerosol solution formulation product comprising actuators with an extremely efficient atomisation in combination with solution formulations consisting substantially of an active ingredient, ethanol and a hydrofluorocarbon as propellant. If a further additive is present in the solution formulation, it is only present in such an amount that it does not have any detrimental influence on the MMAD of the atomised particles.

In one embodiment of the invention the nozzle structure is manufactured as a separate actuator insert piece which is fitted into the nozzle block 14. Alternatively or in addition, the nozzle block may be a separate component fitted into the body portion 10.

Preferably, the actuator insert pieces are constructed of aluminium or stainless steel, as using a CVL to micro drill plastic results in to much heat damage. However, according to one embodiment of the invention it is possible to laser drill into plastics without heat damage, by frequency doubling the visible output of the CVL. This generates three ultra-violet wave lengths, e.g. 255 nm, 271 nm and 289 nm. With these ultra-violet wave lengths plastics can be drilled to high precision without heat damage.

Any kind of actuator inserts known in the art, or of nozzle structures known in the art (e.g. as described in GB-A-2276101 and WO99/12596) can be provided with laser drilled orifices. Preferably, the actuator inserts or nozzle structures are made of aluminium or stainless steel.

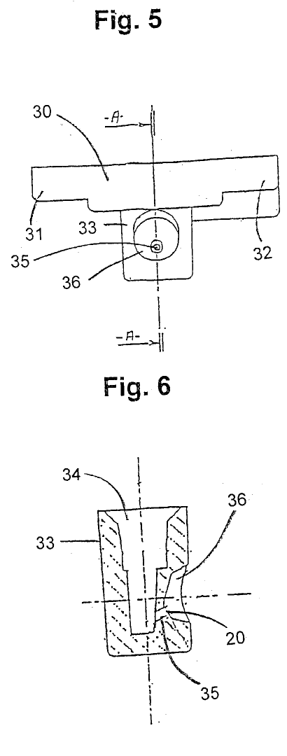

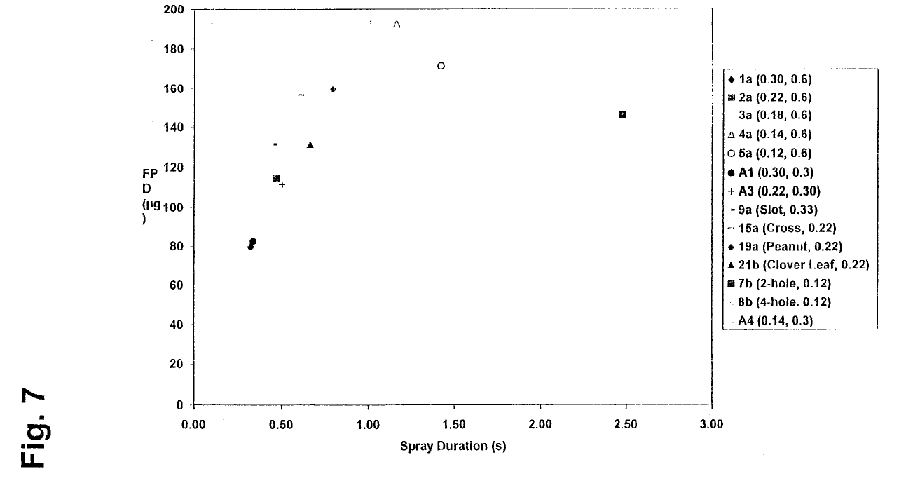

In one embodiment of the present invention an aluminium nozzle block known in the art as the “Chiesi Jet piece” is provided with a laser drilled orifice. FIGS. 5 and 6 show the dimensions of the “Chiesi Jet piece” used in the examples of the present invention. FIG. 5 is a front view of the T shaped nozzle block. FIG. 6 is a section view of the nozzle block along lines A-A of FIG. 5. The “Chiesi Jet piece” is a separate component fitted into the body portion 10. For a detailed description reference is made to international patent application WO99/12596.

The nozzle block (30) is shaped as a T, consisting of an upper bar composed by two fins (31, 32) to be housed and retained in two seats provided in the two shells forming the device and of a vertical stem (33) shorter than the horizontal upper bar.

The vertical stem (33) comprises a socket (34) provided with a seat to house a hollow stem of a pressurized can.

In the thickness of the stem (33) is bored a conduit (35) that connects the socket (34) with the mouth piece (22) of the device through the orifice (20) positioned in a recess (36).

### 3. EXAMPLES

### Example 1

The “Chiesi Jet piece” was used as a model for the aluminium nozzle block in the examples of the present invention. Once drilled the aluminium nozzle block was housed in a modified Bespak 630 series actuator. Test pieces were also constructed and used to check the orifice entrance (inlet) and exit (outlet) diameters. Adjusting the laser power and focus controls the converging and diverging of the orifice. The dimensions of all actuator orifices were checked using a Mitntoyo TM WF20X microscope and Dolan-Jenner Fiberlite.

Table 1 shows the dimensions of a range of actuator orifice diameters from 0.10 mm to 0.18 mm, with 0.60 mm orifice length (n=2). The various shaped orifices that can be produced are slot, cross, clover leaf and peanut having an orifice area comparable to a diameter of 0.10 to 0.18 mm. The dimensions of the peanut are shown in table 2. Multiple holed actuator orifices were also produced. The dimensions of the multiple holed orifices are included in table 2.

TABLE 1
Diameters of the milled actuator inserts
with 0.60 mm orifice length.

| | | | | |
|---|---|---|---|---|
| Orifice diameter (mm) | 0.18 | 0.14 | 0.12 | 0.10 |
| Orifice Length (mm) | 0.60 | 0.60 | 0.60 | 0.60 |

TABLE 2
Diameters of milled actuator inserts with either shaped
or multiple holed orifices (*holes are 0.5 mm apart).

| | | | |
|---|---|---|---|
| Area Cf. (mm) | 0.10 | 0.12 | 0.12 |
| Orifice Shape | Peanut | 2 hole* | 4 hole* |

A high precision can be achieved with laser drilling into aluminium. In table 2 the peanut with an area comparable to a 0.10 mm conventional actuator was produced with two laser drillings.

### Example 2

The experiments of example 2 consisted of discharging beclometasone dipropionate (BDP)/ethanol/HFA 134a formulations, with and without glycerol, through the actuator insert housed in the modified Bespak actuator (630 series) into an Andersen Cascade Impactor operated at 28.3 Lmin⁻¹. Two product strengths, 50 μg/dose (with 7% w/w ethanol, no glycerol) and 250 μg/dose (with 15% w/w ethanol and 1.3% w/w glycerol) were used. The drug deposited on the actuator, the throat and the stages of the impactor were measured. The delivered dose, the mass median aerodynamic diameter (MMAD), the geometric standard deviation (GSD), the fine particle dose ≦4.7 μm (FPD_{≦4.7}) and the fine particle dose ≦1.1 μm (FPD_{≦1.1}) were calculated. The FPDs were also expressed as a % fraction of the ex-valve dose (FPF_{≦4.7}, FPF_{≦1.1}). Shot weight was measured by weighing the pMDI before and after discharge.

The data for the clouds generated by a range of laser drilled orifice diameters all with 0.60 mm length for the 250 μg BDP formula (comparison, with glycerol) and 50 μg BDP formula (according to the invention without glycerol) are given in Table 3a and 3b respectively. Table 4a and 4b gives comparative data generated for the 250 μg and 50 μg BDP formula with the laser drilled orifices with 0.30 mm orifice length.

TABLE 3a
Comparative data generated with BDP 250 μg formula (with
15% w/w ethanol, 1.3% w/w glycerol) and a range of laser
drilled orifice diameters all with 0.60 mm length.

| | | | | | |
|---|---|---|---|---|---|
| Inlet Diameter (mm) | 0.30 | 0.22 | 0.18 | 0.14 | 0.12 |
| Length (mm) | 0.6 | 0.6 | 0.6 | 0.6 | 0.6 |
| Recovered (μg) | 252.22 | 250.11 | 246.84 | 250.96 | 251.38 |
| Delivered (μg) | 235.60 | 232.15 | 233.65 | 13363 | 234.65 |
| Actuator (μg) | 16.63 | 17.94 | 13.17 | 13.88 | 16.74 |
| Throat (μg) | 135.47 | 92.06 | 53.38 | 23.65 | 31.24 |
| Stage 0-2 (μg) | 20.46 | 25.52 | 21.20 | 20.97 | 32.04 |
| Stage 0-2 (%) | 8.11 | 10.2 | 8.59 | 8.36 | 12.75 |
| FPD < 4.7 μm (μg) | 79.67 | 114.59 | 159.1 | 192.47 | 171.37 |
| FPF < 4.7 (%) | 31.59 | 45.82 | 64.45 | 76.69 | 68.17 |
| Dose < 1.1 μm (μg) | 11.93 | 12.62 | 18.44 | 23.27 | 19.79 |
| FPF < 1.1 μm (%) | 4.73 | 5.04 | 7.47 | 9.27 | 7.87 |
| MMAD (μm) | 2.7 | 2.7 | 2.5 | 2.4 | 2.6 |
| GSD | 2.1 | 1.9 | 1.8 | 1.8 | 1.9 |
| Average Shot Weight | 57.8 ± 0.7 | 58.5 ± 0.8 | 57.1 ± 0.7 | 58.1 ± 0.5 | 55.4 ± 2.0 |

TABLE 3b
Data generated with BDP 50 μg formula (with 7% w/w
ethanol, no glycerol) and a range of laser drilled orifice
diameters all with 0.60 mm length.

| | | | | | |
|---|---|---|---|---|---|
| Inlet Diameter (mm) | 0.3 | 0.22 | 0.18* | 0.14* | 0.12* |
| Length (mm) | 0.6 | 0.6 | 0.6 | 0.6 | 0.6 |
| Recovered (μg) | 50.24 | 51.97 | 49.97 | 50.35 | 49.77 |
| Delivered (μg) | 47.05 | 49.00 | 46.65 | 47.00 | 47.10 |
| Actuator (μg) | 3.23 | 2.95 | 3.33 | 3.39 | 2.66 |
| Throat (μg) | 15.49 | 9.07 | 4.45 | 3.50 | 3.01 |
| Stage 0-2 (μg) | 1.33 | 1.62 | 1.11 | 1.22 | 1.47 |
| Stage 0-2 (%) | 2.65 | 3.12 | 2.22 | 2.42 | 2.95 |
| FPD < 4.7 μm (μg) | 30.2 | 38.34 | 41.08 | 42.25 | 42.63 |
| FPF < 4.7 (%) | 60.11 | 73.77 | 82.21 | 83.91 | 85.85 |
| Dose < 1.1 μm (μg) | 14.91 | 19.21 | 24.14 | 27.44 | 24.72 |
| FPF < 1.1 μm (%) | 29.68 | 36.96 | 48.31 | 54.5 | 49.67 |
| MMAD (μm) | 1.1 | 1.1 | 1.0 | 0.9 | 1.0 |
| GSD | 2.0 | 1.9 | 1.9 | 1.9 | 1.9 |
| Average Shot Weight | 59.8 ± 0.7 | 59.6 ± 0.7 | 61.7 ± 0.5 | 60.2 ± 0.5 | 59.4 ± 0.9 |

TABLE 4a
Comparative data generated with BDP 250 μg formula (with 15%
w/w ethanol and 1.3% w/w glycerol) and a range of laser
drilled orifice diameters all with 0.30 mm length.

| | | | |
|---|---|---|---|
| Inlet Diameter (mm) | 0.30 | 0.22 | 0.14 |
| Length (mm) | 0.3 | 0.3 | 0.3 |
| Recovered (μg) | 265.66 | 261.45 | 254.91 |
| Delivered (μg) | 243.05 | 242.80 | 242.65 |
| Actuator (μg) | 22.63 | 18.64 | 12.27 |
| Throat (μg) | 136.75 | 100.16 | 27.58 |
| Stage 0-2 (μg) | 23.65 | 31.34 | 22.18 |
| Stage 0-2 (%) | 8.90 | 11.99 | 8.70 |
| FPD < 4.7 um (μg) | 82.64 | 111.32 | 192.89 |
| FPF < 4.7 (%) | 31.11 | 42.58 | 75.67 |
| Dose < 1.1 μm (μg) | 12.39 | 12.97 | 22.23 |
| FPF < 1.1 μm (%) | 8.66 | 4.96 | 8.72 |
| MMAD (μm) | 2.8 | 3.0 | 2.5 |
| GSD | 2.2 | 2.1 | 1.8 |
| Average Shot Weight | 58.0 ± 1.0 | 58.8 ± 0.5 | 57.7 ± 0.3 |

TABLE 4b
Comparative data generated with BDP 50 μg formula
(with 7% w/w ethanol, no glycerol) and a range of laser
drilled orifice diameters all with 0.30 mm length.

| | | | |
|---|---|---|---|
| Inlet Diameter (mm) | 0.30 | 0.22 | 0.14 |
| Length (mm) | 0.3 | 0.3 | 0.3 |
| Recovered (μg) | 53.92 | 54.59 | 48.76 |
| Delivered (μg) | 48.70 | 50.45 | 45.25 |
| Actuator (μg) | 5.20 | 4.11 | 3.49 |
| Throat (μg) | 17.86 | 14.22 | 7.81 |
| Stage 0-2 (μg) | 2.52 | 3.21 | 4.43 |
| Stage 0-2 (%) | 4.67 | 5.88 | 9.09 |
| FPD < 4.7 um (μg) | 28.34 | 33.06 | 33.03 |
| FPF < 4.7 (%) | 52.56 | 60.56 | 67.74 |
| Dose < 1.1 μm (μg) | 14.90 | 15.17 | 21.35 |
| FPF < 1.1 μm (%) | 27.63 | 27.79 | 43.79 |
| MMAD (μm) | 1.2 | 1.4 | 1.2 |
| GSD | 2.4 | 2.3 | 2.9 |
| Average Shot Weight | 58.3 ± 0.8 | 60.1 ± 0.8 | 59.1 ± 5.05 |

The data generated with the 0.60 mm and 0.30 mm orifice length actuators for the BDP 250 μg and 50 μg formula show a very clear increase in FPF_{≦4.7} as orifice diameter decreases. An optimum orifice diameter/length of 0.14 mm/0.60 mm is seen with the BDP 250 μg formulation giving 76.69% FPF_{≦4.7} and a MMAD of 2.4. However, an improved FPF_{≦4.7} of 83.91% and 85.65% and an improved MMAD of 0.9 and 1.0, respectively, is seen with the BDP 50 μg formulation of the present invention containing no glycerol at an optimum orifice diameter/length of 0.14/0.60 and 0.12/0.60. The increase in FPF_{≦4.7} is accompanied by a decrease in throat deposition and MMAD as orifice diameter decreases. There is very little change in actuator deposition.

Comparisons between the 0.60 mm and 0.30 mm orifice length show no differences for the BDP 250 μg formula in the presence of glycerol. However with the BDP 50 μg formula in the absence of glycerol greater FPF_{≦4.7} and smaller MMAD are achieved with the longer orifice length. For the 0.14 mm diameter orifice a FPF_{≦4.7} of 83.91% is achieved with the 0.60 mm length while a FPF_{≦4.7} of 67.74% is achieved with the 0.30 mm orifice length.

In summary, the actuators of the present invention having an orifice diameter in the range of 0.12 to 0.18 mm and a orifice length of 0.6 mm to 0.8 mm result in combination with a solution formulation being substantially free of low volatility components in an optimisation of the plume characteristics (such as plume duration and fine particle fraction).

### Example 3

The data generated for the peanut orifice shape with the BDP 250 μg and 50 μg formula of Example 2 are shown in table 5a and 5b respectively. The data for the multiple orifice actuator inserts is shown in table 6.

TABLE 5a
Comparison data generated with BDP 250 μg formula.

| Shape | Peanut |
|---|---|
| Area com parable to (mm) | 0.1 |
| Recovered (μg) | 80.14 |
| Delivered (μg) | 11.95 |
| Actuator (μg) | 68.22 |
| Throat (μg) | 3.65 |
| Stage 0-2 (μg) | 1.58 |
| Stage 0-2 (%) | 1.97 |
| FPD < 4.7 μm (μg) | 6.7 |
| FPF < 4.7 (%) | 8.36 |
| Dose < 1.1 μm (μg) | 2.00 |
| FPF < 1.1 μm (%) | 2.50 |
| MMAD (μm) | (2.1) |
| GSD | (2.5) |

TABLE 5b
Data generated with BDP 50 μg formula
(orifice created with two drillings).

| Shape | Peanut |
|---|---|
| Area comparable to (mm): | 0.1 |
| Recovered (μg) | 45.12 |
| Delivered (μg) | 41.2 |
| Actuator (μg) | 3.92 |
| Throat (μg) | 4.92 |
| Stage 0-2 (μg) | 3.8 |
| Stage 0-2 (%) | 8.42 |
| FPD < 4.7 μm (μg) | 32.48 |
| FPF < 4.7 (%) | 71.99 |
| Dose < 1.1 μm (μg) | 18.79 |
| FPF < 1.1 μm (%) | 41.64 |
| MMAD (μm) | 1.2 |
| GSD | 2.7 |

TABLE 6
Data generated with BDP 250 μg and 50 μg formula
and the multiple orifice actuator inserts.

| BDP/Dose | 250 μg | | 50 μg | |
|---|---|---|---|---|
| Shape | 2-holes | 4-holes | 2-holes | 4-holes |
| Area comparable to | 0.12 | 0.12 | 0.12 | 0.12 |
| (mm) | | | | |
| Recovered (μg) | 225.35 | 204.17 | 48.96 | 45.98 |
| Delivered (μg) | 207.97 | 180.95 | 46.05 | 43.65 |
| Actuator (μg) | 17.37 | 23.24 | 2.91 | 2.31 |
| Throat (μg) | 31.77 | 29.25 | 4.12 | 7.53 |
| Stage 0-2 (μg) | 30.42 | 24.58 | 1.97 | 4.34 |
| Stage 0-2 (%) | 13.50 | 12.04 | 4.02 | 9.44 |
| FPD < 4.7 μm (μg) | 145.79 | 127.10 | 39.97 | 31.81 |
| FPF < 4.7 (%) | 64.69 | 62.25 | 81.64 | 69.18 |
| Dose < 1.1 μm (μg) | 16.11 | 17.26 | 20.64 | 16.07 |
| FPF < 1.1 μm (%) | 7.15 | 8.45 | 42.16 | 34.95 |
| MMAD (μm) | 2.7 | 2.5 | 1.2 | 1.4 |
| GSD | 1.9 | 1.9 | 2.0 | 2.7 |
| Average Shot Weight | 52.6 ± 3.0 | 47.1 ± 4.1 | 59.4 ± 0.6 | 56.0 ± 1.6 |

Better results were obtained for the BDP 50 μg formula without glycerol in comparison with the BDP 250 μg formula with glycerol. No additional improvement in FPF_{≦4.7} is achieved with the two and four hole actuator inserts when compared to the single orifice (0.14 mm diameter).

### Example 4

The effect of ethanol content, using the 0.22 mm Bespak actuator as comparator is given in table 7a and 7b for the BDP 250 μg and 50 μg formulations respectively.

The effect of ethanol concentration was assessed using configuration 0.14 mm actuator orifice diameter/0.60 mm orifice length (0.14/0.60) with a 50 μg BDP formula containing 7%, 15% and 25% ethanol. A 250 μg BDP formulation containing 15% and 25% ethanol with and without glycerol was also evaluated. The plume characteristics were assessed visually and the duration of dose generation measured acustically.

TABLE 7a
Effect of percentage of ethanol on atomisation of a BDP 250
μg formula with and without glycerol for 0.14, 0.6 orifice
actuator inserts (*indicates no glycerol in formulation).
0.22 mm Bespak actuator included for comparison

| | Bespak | | | |
|---|---|---|---|---|
| | (0.22, 0.7) | Laser drilled orifice (0.14, 0.6) | | |
| Ethanol (%) | 15 | 15 | 25 | 25* |
| Recovered (μg) | 249.30 | 250.96 | 261.18 | 255.75 |
| Delivered (μg) | 231.90 | 237.10 | 238.75 | 238.85 |
| Actuator (μg) | 18.10 | 13.88 | 22.42 | 16.89 |
| Throat (μg) | 96.20 | 23.65 | 60.11 | 56.58 |
| Stage 0-2 (μg) | 26.60 | 20.97 | 45.68 | 13.35 |
| Stage 0-2 (%) | 10.60 | 8.36 | 17.49 | 5.22 |
| FPD < 4.7 μm (μg) | 108.40 | 192.47 | 133.00 | 168.94 |
| FPF < 4.7 (%) | 43.50 | 76.69 | 50.92 | 66.06 |
| Dose < 1.1 μm (μg) | 12.00 | 23.27 | 11.52 | 36.90 |
| FPF < 1.1 μm (%) | 4.80 | 9.27 | 4.41 | 14.43 |
| MMAD (μm) | 2.9 | 2.4 | 3.3 | 1.8 |
| GSD | 2.0 | 1.8 | 1.9 | 1.9 |
| Average Shot | 58.5 ± 1.5 | 58.1 ± 0.5 | 53.7 ± 0.5 | 54.1 ± 0.6 |
| Weight | | | | |

TABLE 7b
Effect of percentage of ethanol on atomisation of a BDP 50 μg
formula without glycerol for 0.14, 0.6 orifice actuator inserts,
0.22 mm Bespak actuator included for comparison.

| | Bespak 0.22, | Laser drilled orifice (0.14, | |
|---|---|---|---|
| | 0.7 | 0.6) | |
| Ethanol (%) | 7 | 7 | 15 |
| Recovered (μg) | 49.0 | 50.35 | 48.3 |
| Delivered (μg) | 44.9 | 47.0 | 46.2 |
| Actuator (μg) | 4.2 | 3.4 | 2.2 |
| Throat (μg) | 6.7 | 3.5 | 4.9 |
| Stage 0-2 (μg) | 0.9 | 1.2 | 1.2 |
| Stage 0-2 (%) | 1.9 | 2.4 | 2.5 |
| FPD < 4.7 μm (μg) | 37.2 | 42.3 | 40.0 |
| FPF < 4.7 (%) | 76.0 | 83.9 | 82.8 |
| Dose < 1.1 μm (μg) | 22.4 | 27.4 | 18.6 |
| FPF < 1.1 μm (%) | 45.8 | 54.5 | 38.4 |
| MMAD (μm) | 0.9 | 0.9 | 1.2 |
| GSD | 1.9 | 1.9 | 1.8 |
| Average Shot Weight | 58.7 ± 0.3 | 60.2 ± 0.5 | 57.5 ± 0.3 |

The 0.14, 0.6 orifice actuator insert was used to evaluate the effect of increasing the percentage of ethanol in the BDP 250 μg formula and 50 μg formula. Even with 25% ethanol in the BDP 250 μg formula the 50.9% FPF_{≦4.7} obtained is greater than the FPF_{≦4.7} obtained with the 15% ethanol formula and a 0.22 mm conventional Bespak actuator. However, as a consequence of increasing the ethanol content the MMAD obtained is also increased. This can be corrected by removing or altering the percentage of glycerol (or in general the low volatility component) in the formula. This gives the formulator great scope for manipulating the formulation and achieving high drug loading and efficient atomisation if required. This is further demonstrated by the BDP 50 μg results (without glycerol) where no loss in FPF_{≦4.7} is seen when the ethanol content is increased from 7 to 15%.

### Example 5

Through life testing by carrying out Andersen Cascade Impactor determinations for shots 6-15 and shots 191-200 was also carried out to evaluate actuator blockage.

The results of the through life tests with no actuator cleaning are shown in table 8.

TABLE 8
Through life testing with no actuator cleaning on drilled
actuator inserts with a BDP 250 μg formulation.

| | 15% Ethanol, 1.3% Glycerol | | | | 25% Ethanol | |
|---|---|---|---|---|---|---|
| Orifice | 0.14, 0.6 | | 0.18, 0.6 | | 0.14, 0.6 | |
| Shots | 6-15 | 191-200 | 6-15 | 191-200 | 6-15 | 191-200 |
| Recovered (μg) | 244.83 | 226.63 | 237.06 | 243.51 | 254.87 | 234.02 |
| Delivered (μg) | 233.00 | 215.20 | 226.70 | 237.70 | 238.10 | 222.90 |
| Actuator (μg) | 11.83 | 11.44 | 10.35 | 5.81 | 16.74 | 11.12 |
| Throat (μg) | 25.09 | 32.92 | 41.36 | 65.52 | 56.11 | 55.06 |
| Stage 0-2 (μg) | 25.61 | 60.88 | 25.24 | 27.34 | 13.96 | 21.99 |
| Stage 0-2 (%) | 10.46 | 26.86 | 10.65 | 11.23 | 5.48 | 9.40 |
| FPD < 4.7 μm (μg) | 182.30 | 121.39 | 160.10 | 144.84 | 168.06 | 145.85 |
| FPF < 4.7 (%) | 74.46 | 53.56 | 67.54 | 59.48 | 65.94 | 62.32 |
| Dose < 1.1 μm (μg) | 16.92 | 9.44 | 14.60 | 16.51 | 36.02 | 29.07 |
| FPF < 1.1 μm (%) | 6.91 | 4.17 | 6.16 | 6.78 | 14.13 | 12.42 |
| MMAD (μm) | 2.6 | 3.5 | 2.7 | 2.6 | 1.8 | 2.1 |
| GSD | 1.8 | 1.9 | 1.8 | 1.8 | 1.9 | 2.4 |
| Average Shot Weight | 55.4 ± 0.5 | 55.5 ± 1.6 | 55.9 ± 0.3 | 55.2 ± 0.9 | 54.2 ± 0.9 | 52.2 ± 0.7 |

Through life testing was conducted with the 250 μg BDP formula. It is clear from the results that the efficiency of atomisation is decreased at the end of the can life with the 0.14, 0.6 orifice insert. A major increase in the MMAD is also observed. The results obtained with the formula containing 25% ethanol and no glycerol shows a small increase in the MMAD and GSD. However no change in FPF_{≦4.7} is seen between beginning and end of can life. The results obtained with the 0.18, 0.6 insert show only a small decrease in efficiency through life.

### Example 6

Finally, the relation of spray (plume) duration and fine particle dose for laser drilled actuator inserts generated with BDP 250 μg formulation with 15% w/w ethanol and 1.3% w/w glycerol is given in FIG. 7. The term “(0.14, 0.6)” describes an actuator with an actuator orifice diameter of 0.14 mm and an orifice length of 0.6 mm.

In summary, it can be concluded that orifice diameter decrease and length increase combine to produce fine sprays. FIG. 7 clearly shows that spray duration increases as orifice diameter decreases. A spray duration of over one second can be produced with an orifice of 0.14 mm diameter and 0.6 mm length, with no loss in the FPF_{≦4.7} obtained.

Thus, the present invention confirms that changes in diameter and length of actuator orifices influence the speed (duration) and fine particle characteristics of clouds.

### Example 7

In example 7, the actuator blockage/device clogging has been tested for beclometasone dipropionate (BDP, 250 μg) solution formulations with and without a low volatility component (LVC).

Table 9 shows the results of an actuator test.

TABLE 9
Effect of the low volatility component on actuator
blockage (actuator orifice length = 0.6 mm)

| | | | Orifice | | | |
|---|---|---|---|---|---|---|
| | | | Diameter | | | |
| | % | | Inlet | Outlet | Actuator | Pass/ |
| Drug | EtOH | % LVC | (mm) | (mm) | material | Fail |
| BDP 250 | 15 | 1.3% | 0.14 | 0.14 | Aluminium | Fail |
| | | Glycerol | | | | |
| BDP 250 | 15 | 0% Glycerol | 0.14 | 0.14 | Aluminium | Pass |

Actuator orifice length of the nozzle blocks of the present invention refers to the distance between the external face (outlet) and the internal surface (inlet) which due to the design of the nozzle blocks are preferably parallel.

According to the results shown in table 9, the presence of 1.3% glycerol resulted in actuator blockage for an actuator made of aluminium and having an inlet and outlet orifice diameter of 0.14 mm. On the other hand, a corresponding solution formulation containing no glycerol passed the actuator blockage test.

### Example 8

In Example 8, the influence of the presence of a low volatility component in a solution formulation containing dexbudesonide in 17% by weight ethanol has been tested for an actuator with an orifice diameter of 0.14 mm and an orifice length of 0.6 mm. The FPF_{≦4.7 μm} and MMAD have been determined (Table 10).

TABLE 10

| | | | Orifice | | MMAD |
|---|---|---|---|---|---|
| Drug Dose (μg) | Ethanol (EtOH) % | Glycerol % | diameter mm | FPF_{≦4.7 μm} % | μm |
| Dexbudesonide | 17 | 0.3 | 0.14 | 82.8 | 1.8 |
| 160 | | | | | |
| Dexbudesonide | 17 | 1.3 | 0.14 | 70.1 | 2.9 |
| 160 | | | | | |

According to the results shown in table 10, an increase of the amount of low volatility component (glycerol) results in a lower FPF_{≦4.7 μm} and a higher MMAD. Accordingly, a low content of 0 to 0.5%, preferably 0 to 0.3% low volatility component in the solution formulation not only has a beneficial effect with respect to blockage problems of the actuator, however, in addition, the FPF_{≦4.7 μm} and the MMAD is improved considerably.

Additional results have been obtained with 80 μg dexbudesonide HFA solution formulation comprising 15% by weight ethanol and 2% by weight water delivered through a 0.14 mm diameter and 0.7 mm length laser drilled orifice that gives a FPF of over 75%. The same formulation provided with a conventional 0.22 mm Bespak actuator gives a FPF of 45%.

The data has revealed major new insights in the use of pMDI. Extremely efficient atomisation can be achieved with formulations containing high levels of ethanol and with a high ratio of ethanol to active ingredient and being substantially free of low volatility components such as glycerol. No loss of atomisation efficiency is seen with formulations containing up to 15% ethanol. FPF_{≦4.7} of over 50% can be achieved with formulations containing 25% ethanol. This allows the use of poorly soluble active ingredients in HFA solution formulations having a high ethanol content in order to transfer the poorly soluble active ingredient in solution. Accordingly, the present invention allows the use of new solution formulations also with poorly soluble active ingredients, which was not possible before the present invention was made.

Moreover, the data demonstrates that formulations previously unsuitable for pulmonary delivery (7% or more ethanol with a ratio of ethanol to active ingredients of at least 20:1, 0 to about 0.5% glycerol) when used with the small diameter drilled inserts can produce highly efficient sprays with a much smaller MMAD, reduced throat and actuator deposition, while actuator blockage and clogging problems can be avoided.

Other preferred formulations which can be used according to the present invention are the following:

| | |
|---|---|
| Formulation 1: | |
| Salmeterol xinafoate | 3 mg/can (˜0.025% w/v) |
| Ethanol | 30% (w/w) |
| Water | 3% (w/w) |
| UFA 134a | 67% (w/w) |
| Formulation 2: | |
| Fluticasone propionate | 15 mg/can (˜0.12% w/v) |
| Ethanol | 30% (w/w) |
| Water | 3% (w/w) |
| HFA 134a | 67% (w/w) |
| Formulation 3: | |
| Mometasone propionate | 6 mg/can (˜0.05% w/v) |
| Ethanol | 30% (w/w) |
| Water | 3% (w/w) |
| HFA 134a | 67% (w/w) |

The formulation is actuated by a metering valve capable of delivering a volume of between 50 μl and 100 μl.

The choice of the metering valve and type will be made according the knowledge of the person skilled in the art.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A pressurized metered dose inhaler actuator comprising:
an expansion chamber; and
a nozzle block, said nozzle block having an actuator orifice opening into said expansion chamber, said actuator orifice having a diameter in the range of from about 0.10 mm to about 0.20 mm over the entire length of the actuator orifice, and said actuator orifice having a length in the range of from about 0.60 mm to about 1.0 mm.

2. An actuator as defined in claim 1, wherein the diameter of the actuator orifice is in the range of 0.11 mm to 0.18 mm.

3. An actuator as defined in claim 1, wherein the diameter of the actuator orifice is 0.12 mm.

4. An actuator as defined in claim 1, wherein the diameter of the actuator orifice is 0.14 mm.

5. An actuator as defined in claim 1, wherein the diameter of the actuator orifice is 0.16 mm.

6. An actuator as defined in claim 1, wherein the diameter of the actuator orifice is 0.18 mm.

7. An actuator as defined in claim 1, wherein the length of the actuator orifice is in the range of 0.6 mm to 0.8 mm.

8. An actuator as defined in claim 1, wherein the actuator orifice has the shape of a slot, cross, clover leaf or peanut.

9. An actuator as defined in claim 1, wherein the nozzle block contains more than one actuator orifice.

10. An actuator as defined in claim 1, wherein the nozzle block if constructed of aluminum or stainless steel.

11. An actuator as defined in claim 1, wherein the actuator orifice is formed using a laser.

12. A medicinal aerosol device, comprising:
a pressurized metered dose inhaler including an actuator having an expansion chamber and a nozzle block, said nozzle block being provided with an actuator orifice opening into said expansion chamber, the actuator orifice having a diameter in the range of from about 0.10 mm to about 0.20 mm over the entire length of the actuator orifice, and the actuator orifice having a length in the range of from about 0.60 mm to about 1.0 mm; and
a medicinal aerosol solution including an active ingredient, a hydrofluorocarbon propellant, 7% (w/w) or more ethanol as a co-solvent, based on the solution formulation, and wherein the ratio of ethanol:active ingredient is at least 20:1.

13. A medicinal aerosol device as defined in claim 12, further comprising a low volatility component in an amount of from 0 to 0.5% by weight.

14. A medicinal aerosol device as defined in claim 12, wherein the medicinal aerosol solution contains at least 15% (w/w) ethanol.

15. A medicinal aerosol device as defined in claim 12, wherein the medicinal aerosol solution contains at least 20% (w/w) ethanol.

16. A medicinal aerosol device as defined in claim 12, wherein the active ingredient is a corticosteroid selected from the group consisting of beclometasone dipropionate, budesonide, dexbudesonide, ciclesonide, fluticasone propionate and mometasone propionate, or a β₂-agonist selected from the group consisting of formoterol, salmeterol xinafoate and TA 2005.

17. A medicinal aerosol device as defined in claim 12, wherein the low volatility component is selected from the group consisting of glycerol, propylene glycol, polyethylene glycol and isopropylmyristate.

18. A medicinal aerosol device as defined in claim 12, wherein the propellant is selected from the group consisting of HFA227, HFA134a, and their mixtures.

19. A method for forming an actuator orifice in a nozzle block for use in a pressurized metered dose inhaler actuator, comprising the steps of:
obtaining a nozzle block into which an orifice is to be formed; and
forming an actuator orifice in said nozzle block using a laser.
